(19) United States
(12) Patent Application Publication
(10) Pub. No.: US 2026/0202392 A1
(43) Pub. Date: Jul. 16, 2026

(54) DUAL FREQUENCY COMB SPECTROSCOPY FOR MEDICINAL FLUID ANALYSIS

(71) Applicant: Honeywell International Inc., Charlotte, NC (US)

(72) Inventors: Daniel James YEE, Charlotte, NC (US); Moin S. SHAFAI, Plano, TX (US); Andy Walker BROWN, Richardson, TX (US); Richard A. WADE, Worthington, OH (US)

(21) Appl. No.: 19/416,817

(22) Filed: Dec. 11, 2025

Related U.S. Application Data
(60) Provisional application No. 63/744,014, filed on Jan. 10, 2025.

(51) Int. Cl. G01N 33/15; G01N 21/03; G01N 21/25; G01N 21/31; G01N 21/85

(52) U.S. Cl. CPC G01N 33/15; G01N 21/0303; G01N 21/255; G01N 21/31; G01N 21/3103; G01N 21/85

(57) ABSTRACT

The present disclosure provides a device for in situ spectroscopic analysis of medicinal fluid. The device comprises a positioning structure, a dual frequency comb (DFC) on a first side, a photodetector on the opposite side facing the DFC, and a processing element. The positioning structure positions the DFC and photodetector at desired separation distance on opposite sides of a medicinal fluid delivery container. The DFC emits light passing through the container and medicinal fluid. The photodetector receives the emitted light from the DFC that has been exposed to elements within the medicinal fluid exposed to the emitted light. The processing element generates an optical absorption spectrum from the received light and identifies elements within the medicinal fluid.

### CROSS-REFERENCE TO RELATED APPLICATIONS

This application claims priority to U.S. Provisional Patent Application No. 63/744,014, titled SPECTROSCOPIC SYSTEM AND METHOD FOR ANALYZING COMPOUNDS IN INTRAVENOUS BAGS, filed January 10, 2025, which is hereby incorporated by reference in its entirety.

### FIELD OF INVENTION

The present disclosure relates to spectroscopic analysis, and more particularly to spectroscopic analysis systems for medical applications.

### BACKGROUND

Medicinal fluid administration in healthcare settings requires precise dosing and accurate identification of pharmaceutical compounds to ensure patient safety and therapeutic efficacy. Intravenous (IV) delivery systems are commonly used to administer various medications, including chemotherapy agents, antibiotics, pain medications, and other therapeutic compounds directly into a patient's bloodstream. These delivery systems typically utilize flexible containers such as IV bags that hold the medicinal fluid and connect to tubing systems for controlled administration.

Traditional methods for verifying the contents and concentrations of medicinal fluids in IV containers rely primarily on visual inspection of labels and manual verification procedures. However, these approaches may not detect formulation errors, contamination, degradation of active compounds, or other quality issues that could affect patient outcomes. Additionally, certain medications may undergo chemical changes over time or under specific storage conditions, potentially altering their therapeutic properties or creating harmful byproducts.

The inventors have identified numerous areas of improvement in the existing technologies and processes, which are the subjects of embodiments described herein. Through applied effort, ingenuity, and innovation, many of these deficiencies, challenges, and problems have been solved by developing solutions that are included in embodiments of the present disclosure, some examples of which are described in detail herein.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

Various embodiments described herein relate to systems, devices, and methods for in situ spectroscopic analysis of a medicinal fluid.

According to an aspect of the present disclosure, a device for in situ spectroscopic analysis of a medicinal fluid is provided. The device comprises a positioning structure. The device comprises a dual frequency comb (DFC) positioned on a first side of the positioning structure. The device comprises a photodetector positioned on a second, opposite side of the positioning structure and facing the DFC. The device comprises at least one processing element. The positioning structure is adapted to position the DFC and the photodetector at a desired separation distance on opposite sides of a medicinal fluid delivery container containing a medicinal fluid. The DFC is adapted to emit light that passes through the medicinal fluid delivery container and the medicinal fluid. The photodetector is adapted to receive the emitted light from the DFC that has been exposed to one or more elements within the medicinal fluid exposed to the emitted light. The at least one processing element is adapted to generate an optical absorption spectrum from the received light from the photodetector and identify at least one of the one or more elements within the medicinal fluid exposed to the emitted light.

According to other aspects of the present disclosure, the device may include one or more of the following features. The desired separation distance may be one millimeter or less. The DFC may comprise a photonic integrated circuit (PIC)-scale DFC. The DFC may comprise a fiber-based DFC. The medicinal fluid delivery container may comprise an intravenous (IV) bag. The positioning structure may be adjustable to vary the separation distance between the DFC and the photodetector. The medicinal fluid delivery container may comprise tubing in fluid connection with an IV bag. The medicinal fluid may comprise one or more cancer treatment medicine. The one or more cancer treatment medicines may comprise one or more of a chemotherapy agent, an immunotherapy agent, a targeted therapy agent, and/or a hormonal therapy agent. A wavelength range of the light emitted by the DFC may be selected based a desired one or more cancer treatment medicines to be analyzed.

According to another aspect of the present disclosure, a method for in situ spectroscopic analysis of a medicinal fluid is provided. The method comprises positioning, using a positioning structure, a dual frequency comb (DFC) on a first side of a medicinal fluid delivery container and a photodetector on a second, opposite side of the medicinal fluid delivery container containing a medicinal fluid with a desired separation distance therebetween. The method comprises emitting light from the DFC such that the emitted light passes through the medicinal fluid. The method comprises receiving, by the photodetector, the emitted light from the DFC that has been exposed to one or more elements within the medicinal fluid exposed to the emitted light. The method comprises generating, by at least one processing element, an optical absorption spectrum from the received light from the photodetector. The method comprises identifying, by the at least one processing element, at least one of the one or more elements within the medicinal fluid exposed to the emitted light.

According to other aspects of the present disclosure, the method may include one or more of the following features. The desired separation distance may be one millimeter or less. The DFC may comprise a photonic integrated circuit (PIC)-scale DFC. The DFC may comprise a fiber-based DFC. The medicinal fluid delivery container may comprise an intravenous (IV) bag. The method may further comprise adjusting the positioning structure to vary the separation distance between the DFC and the photodetector. The medicinal fluid delivery container may comprise tubing in fluid connection with an IV bag. The medicinal fluid may comprise one or more cancer treatment medicine. The one or more cancer treatment medicines may comprise one or more of a chemotherapy agent, an immunotherapy agent, a targeted therapy agent, and/or a hormonal therapy agent. A wavelength range of the light emitted by the DFC may be selected based a desired one or more cancer treatment medicines to be analyzed.

The foregoing general description of the illustrative embodiments and the following detailed description thereof are merely exemplary aspects of the teachings of this disclosure and are not restrictive.

### BRIEF DESCRIPTION OF FIGURES

The description of the illustrative embodiments is read in conjunction with the accompanying figures. It will be appreciated that, for simplicity and clarity of illustration, elements illustrated in the figures have not necessarily been drawn to scale, unless described otherwise. For example, the dimensions of some of the elements is exaggerated relative to other elements, unless described otherwise. Embodiments incorporating teachings of the present disclosure are shown and described with respect to the following figures presented herein.

FIG. 1 illustrates an isometric view of an IV bag with multiple access ports and sampling chamber types, according to aspects of the present disclosure.

FIG. 2 illustrates a detailed view of a sampling configuration with a dual frequency comb and photodetector, according to aspects of the present disclosure.

FIG. 3 illustrates an isometric view of the IV bag of FIG. 1 with dual positioning mechanisms for spectroscopic analysis, according to aspects of the present disclosure.

FIG. 4 illustrates a cross-sectional view of the IV bag of FIG. 1 positioned within a positioning mechanism, according to aspects of the present disclosure.

FIG. 5 illustrates a front orthogonal view of the IV bag of FIG. 1 positioned within a positioning mechanism and ready for analysis, according to aspects of the present disclosure.

FIG. 6 illustrates a block diagram of an analysis system for analyzing fluid medicine using dual frequency comb spectroscopy, according to aspects of the present disclosure.

### DETAILED DESCRIPTION

Some embodiments of the present disclosure will now be described more fully hereinafter with reference to the accompanying drawings, in which some, but not all embodiments of the disclosure are shown. Indeed, these disclosures are embodied in many different forms and should not be construed as limited to the embodiments set forth herein; rather, these embodiments are provided so that this disclosure will satisfy applicable legal requirements. Like numbers refer to like elements throughout.

As used herein, terms such as “front,” “rear,” “top,” “bottom,” “left,” “right,” etc. are used for explanatory purposes in the examples provided below to describe the relative position of certain components or portions of components. Furthermore, as would be evident to one of ordinary skill in the art in light of the present disclosure, the terms “substantially” and “approximately” indicate that the referenced element or associated description is accurate to within applicable engineering tolerances.

As used herein, the term “comprising” means including but not limited to and should be interpreted in the manner it is typically used in the patent context. Use of broader terms such as comprises, includes, and having should be understood to provide support for narrower terms such as consisting of, consisting essentially of, and comprised substantially of.

The phrases “in one embodiment,” “according to one embodiment,” “in some embodiments,” and the like generally mean that the particular feature, structure, or characteristic following the phrase is included in at least one embodiment of the present disclosure and is included in more than one embodiment of the present disclosure (importantly, such phrases do not necessarily refer to the same embodiment).

The phrases “in one example,” “according to one example,” “in some examples,” and the like generally mean that the particular feature, structure, or characteristic following the phrase is included in at least one example of the present disclosure and is included in more than one example of the present disclosure (importantly, such phrases do not necessarily refer to the same example).

If the specification states a component or feature “may,” “can,” “could,” “should,” “would,” “preferably,” “possibly,” “typically,” “optionally,” “for example,” “as an example,” “in some examples,” “often,” or “might” (or other such language) be included or have a characteristic, that specific component or feature is not required to be included or to have the characteristic. Such component or feature is optionally included in some examples, or it is excluded.

The word “example” or “exemplary” is used herein to mean “serving as an example, instance, or illustration.” Any implementation described herein as “example” or “exemplary” is not necessarily to be construed as preferred or advantageous over other implementations.

The term “electronically coupled,” “electronically coupling,” “electronically couple,” “in communication with,” “in electronic communication with,” or “connected” in the present disclosure refers to two or more elements or components being connected through wired means and/or wireless means, such that signals, electrical voltage/current, data and/or information is transmitted to and/or received from these elements or components.

The term “component” may refer to an article, a device, or an apparatus that may comprise one or more surfaces, portions, layers and/or elements. For example, an example component may comprise one or more substrates that provide underlying layer(s) for the component and may comprise one or more elements that may form part of and/or are disposed on top of the substrate. In the present disclosure, the term “element” may refer to an article, a device, or an apparatus that may provide one or more functionalities.

The term “in fluid communication” refers to two or more components being connected, directly or indirectly, so that a fluid (liquid or gas) can flow between them.

Dual frequency comb spectroscopy represents an advanced optical technique for analyzing medicinal fluids within intravenous delivery containers. The technology employs two optical frequency combs with slightly different repetition rates to generate a broadband spectrum of precisely spaced optical frequencies. When these optical frequency combs interact with a sample, the resulting interferometric signals provide high-resolution spectral information that enables identification and quantification of molecular components within the medicinal fluid.

The spectroscopic analysis approach utilizes the principle that different molecular compounds absorb light at characteristic wavelengths. By directing light from dual frequency combs through a medicinal fluid sample, the transmitted or reflected light carries spectral signatures that correspond to the molecular composition of the fluid. The detection system captures these optical signals and processes the spectral data to determine the presence and concentration of specific compounds within the medicinal fluid.

In medicinal fluid analysis applications, dual frequency comb spectroscopy may provide real-time monitoring capabilities for verifying drug concentrations, detecting contamination, and ensuring proper formulation of therapeutic compounds. The technology may enable non-invasive in situ analysis through container materials or may utilize sampling configurations that maintain the sterility and integrity of the medicinal fluid during analysis. In this regard, the analyzed fluid is not wasted.

The spectroscopic system may incorporate processing elements that compare detected spectral signatures against reference databases to identify specific pharmaceutical compounds. The analysis may determine concentration levels of active pharmaceutical ingredients, excipients, and potential contaminants within the medicinal fluid. The processing elements may generate reports indicating the composition and quality of the analyzed medicinal fluid for healthcare providers and pharmacy personnel.

Dual frequency comb spectroscopy may offer advantages over conventional analytical methods by providing rapid analysis times, high spectral resolution, and the capability to simultaneously detect multiple compounds within a single measurement. The technology may enable point-of-care analysis without requiring sample preparation or laboratory processing, thereby reducing analysis time and maintaining the chain of custody for medicinal fluids.

A system for in situ spectroscopic analysis of a medicinal fluid may employ a fluid sampling chamber configuration that enables controlled analysis of medicinal compounds without compromising the sterility or integrity of the primary medicinal fluid supply. The system may include a fluid sampling chamber in fluid communication with a medicinal fluid delivery container (e.g., IV bag) to receive a sample of a medicinal fluid from the medicinal fluid delivery container. The fluid sampling chamber may provide a controlled optical path for spectroscopic analysis while maintaining fluid communication with the medicinal fluid delivery container.

The fluid sampling chamber may comprise a transparent or translucent structure that allows optical transmission while containing the medicinal fluid sample. In some cases, the fluid sampling chamber includes inlet and outlet ports that enable fluid transfer between the medicinal fluid delivery container and the sampling chamber. The fluid communication may be established through tubing, connectors, or direct attachment mechanisms that maintain a sterile pathway for medicinal fluid transfer.

A dual frequency comb (DFC) may be positioned on a first side of the fluid sampling chamber to emit light through the sample of the medicinal fluid within the fluid sampling chamber. The DFC may generate coherent optical radiation across a broad spectral range with precisely controlled frequency spacing. The positioning of the DFC on the first side of the fluid sampling chamber may enable direct optical coupling of the emitted light into the medicinal fluid sample contained within the sampling chamber.

The system may include a photodetector positioned on a second, opposite side of the fluid sampling chamber to receive the emitted light from the DFC that has been exposed to one or more elements within the sample of the medicinal fluid within the fluid sampling chamber. The photodetector may be configured to receive the emitted light from the DFC that has been exposed to the medicinal fluid.

At least one processing element may be included for generating an optical absorption spectrum from the received light from the photodetector and identifying at least one of the one or more elements within the fluid sampling chamber exposed to the emitted light. The processing element may receive electrical signals from the photodetector and apply signal processing algorithms to extract spectral information from the received light. The processing element may correlate the received light with the optical wavelengths emitted by the DFC to reconstruct an absorption spectrum of the medicinal fluid sample.

The processing element may compare the generated optical absorption spectrum against reference spectral databases to identify specific molecular components within the medicinal fluid. The identification process may involve pattern matching algorithms, spectral deconvolution techniques, or machine learning approaches that recognize characteristic absorption features of pharmaceutical compounds. The processing element may determine the presence and concentration of active pharmaceutical ingredients, excipients, or contaminants within the medicinal fluid sample based on the spectral analysis results.

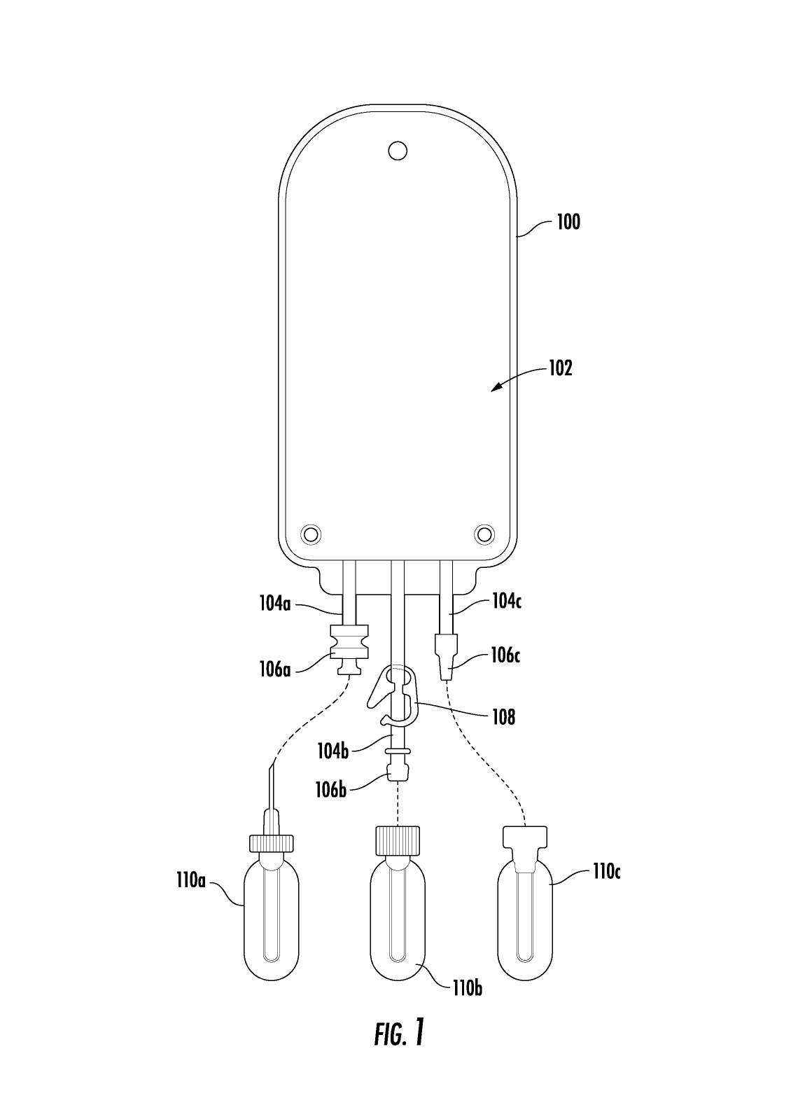

Referring to FIG. 1, an IV bag 100 may contain fluid medicine 102 and may be configured with multiple access interfaces for connection to fluid sampling chambers. The IV bag 100 may comprise a flexible container constructed from medical-grade materials that maintain the sterility and integrity of the fluid medicine 102 during storage and administration. The fluid medicine 102 may include various therapeutic compounds such as chemotherapy agents, immunotherapy agents, targeted therapy agents, hormonal therapy agents, or other pharmaceutical formulations requiring precise concentration verification.

The IV bag 100 may include a first access port 104a, a second access port 104b, and/or a third access port 104c positioned to enable fluid communication with delivery devices and sampling systems. Each access port may provide a sterile interface for connecting tubing, syringes, or sampling devices to access the fluid medicine 102 contained within the IV bag 100. The multiple access ports may enable simultaneous or sequential connections for different purposes, such as fluid administration, sampling, or monitoring applications.

The first access port 104a may be coupled to a first connector type 106a, the second access port 104b may be coupled to a second connector type 106b, and the third access port 104c may be coupled to a third connector type 106c. The different connector types may provide compatibility with various medical devices and sampling systems. In some cases, the connector types may include luer lock connectors, luer slip connectors, needle-free connectors, or proprietary connection interfaces designed for specific medical applications. The first connector type 106a, second connector type 106b, and third connector type 106c may enable secure and sterile connections while preventing accidental disconnection during fluid transfer operations. A clamp 108 may be positioned on one or more of the access ports to selectively stop the flow of the fluid medication through that respective access port.

The system may include a first sampling chamber type 110a, a second sampling chamber type 110b, and a third sampling chamber type 110c that may be configured to interface with the respective access ports and connector types. Each sampling chamber type may comprise a transparent or translucent structure that enables optical analysis of fluid medicine 102 samples while maintaining sterile fluid pathways. The first sampling chamber type 110a may be configured for connection to the first access port 104a via the first connector type 106a, the second sampling chamber type 110b may be configured for connection to the second access port 104b via the second connector type 106b, and the third sampling chamber type 110c may be configured for connection to the third access port 104c via the third connector type 106c.

The fluid sampling chambers may be selectively connectable to one of the access ports of the IV bag 100 via the connectors and valves associated with each access port. The selective connection capability may enable healthcare providers to choose appropriate sampling configurations based on the specific analysis requirements or the type of fluid medicine 102 being analyzed. In some cases, the fluid sampling chamber may be attached to the IV bag 100 using luer locks and syringes to extract medication from the IV bag and push the medication into the sampling chamber.

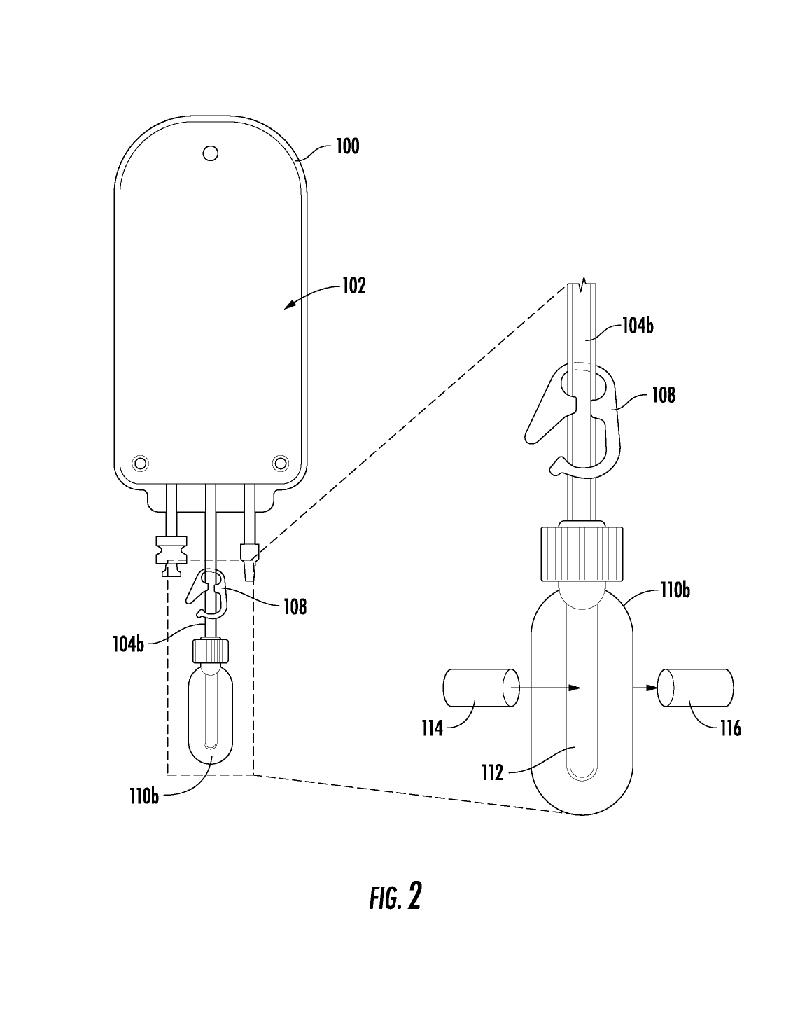

The sampling chamber types may comprise imaging windows that provide controlled optical path lengths for spectroscopic analysis. The imaging windows may be positioned within the fluid sampling chambers to enable optical transmission through fluid medicine 102 samples while maintaining precise geometric relationships for accurate spectral measurements. The DFC and photodetector components may be aligned with the imaging windows to ensure proper optical coupling and signal detection during spectroscopic analysis procedures. In particular, the distance between the DFC and the photodetector must be a fixed, known distance. In the embodiment of FIGS. 1 and 2, the thickness of the sampling chamber provides the fixed, known distance.

Referring to FIG. 2, a detailed sampling configuration may be implemented using the second sampling chamber type 110b to enable controlled spectroscopic analysis of the fluid medicine 102. In FIG. 2, the second sampling chamber type 110b is positioned in-line with the fluid path from the second access port 104b of the IV bag 100. The second sampling chamber type 110b includes a connection point only at the top end to interface with tubing from the second access port 104b, enabling fluid transfer between the IV bag 100 and the sampling chamber 110b and back to the IV bag 100 so that the fluid medicine is not wasted.

An imaging window 112 may be incorporated within the second sampling chamber type 110b to provide a transparent optical interface through which spectroscopic analysis may be performed. The imaging window 112 may comprise a transparent material such as glass, quartz, or optical-grade plastic that enables optical transmission while maintaining the structural integrity of the fluid sampling chamber. The imaging window 112 may be positioned within the second sampling chamber type 110b to create a controlled optical path length through the fluid medicine 102 sample contained within the sampling chamber.

A dual frequency comb (DFC) 114 is positioned on one side of the imaging window 112 to emit light through the fluid medicine 102 contained within the imaging window 112. The DFC 114 may generate coherent optical radiation across a broad spectral range with precisely controlled frequency spacing between adjacent spectral lines. The positioning of the DFC 114 adjacent to the imaging window 112 may enable direct optical coupling of the emitted light into the fluid medicine 102 sample for spectroscopic analysis.

The DFC 114 may comprise a photonic integrated circuit (PIC)-scale DFC that provides compact optical frequency generation capabilities. The PIC-scale DFC may integrate multiple optical components on a single semiconductor substrate, enabling miniaturized dual frequency comb generation suitable for portable or point-of-care analysis applications. In some cases, the DFC 114 may comprise a fiber-based DFC that utilizes optical fiber components to generate and manipulate optical frequency combs. The fiber-based DFC may provide enhanced spectral coverage and power handling capabilities compared to chip-scale implementations.

A photodetector 116 is positioned on the opposite side of the imaging window 112 to receive and detect the transmitted light from the DFC 114. The photodetector 116 may be aligned with the DFC 114 to ensure proper optical coupling and signal detection during spectroscopic analysis procedures. The photodetector 116 may comprise photodiodes, avalanche photodiodes, or other optical detection elements capable of converting optical signals into electrical signals for subsequent processing.

The arrangement of the DFC 114 and photodetector 116 on opposing sides of the imaging window 112 may enable spectroscopic analysis of the fluid medicine 102 as the fluid medicine 102 passes through or is held within the second sampling chamber type 110b. The optical path through the imaging window 112 may provide a controlled distance for light transmission through the fluid medicine 102 sample, enabling accurate measurement of optical absorption characteristics.

The active sampling distances between the DFC 114 and photodetector 116 may be less than 1 mm to ensure proper analysis of the fluid medicine 102. The sub-millimeter optical path length may provide adequate optical interaction with the fluid medicine 102 while minimizing signal attenuation and maintaining sufficient signal-to-noise ratios for accurate spectral measurements. The controlled optical path length may enable detection of trace concentrations of compounds within the fluid medicine 102.

The fluid medicine 102 may comprise a cancer treatment medicine that requires precise concentration verification and quality control. The cancer treatment medicine may comprise one or more of a chemotherapy agent, an immunotherapy agent, a targeted therapy agent, or a hormonal therapy agent. The spectroscopic analysis configuration may enable real-time verification of drug concentrations and detection of potential degradation products or contaminants within the cancer treatment medicine formulations.

A method for in situ spectroscopic analysis of a medicinal fluid may involve a series of coordinated steps that enable controlled sampling, analysis, and fluid recovery while maintaining sterility and measurement accuracy. The method may begin with transferring a sample of a medicinal fluid from a medicinal fluid delivery container to a fluid sampling chamber in fluid communication with the medicinal fluid delivery container. The transfer process may utilize sterile fluid pathways that preserve the integrity of the medicinal fluid while enabling controlled sampling for spectroscopic analysis.

The fluid transfer may be accomplished through syringe-based mechanisms that provide precise control over sample volumes and fluid movement. The one or more connectors and valves may be configured to enable the medicinal fluid to be drawn from the medicinal fluid delivery container via a syringe and pushed into the fluid sampling chamber via the syringe. The syringe-based transfer system may provide healthcare providers with direct control over the sampling process while maintaining sterile conditions throughout the fluid handling procedure.

The method may proceed with emitting light from a dual frequency comb (DFC) positioned on a first side of the fluid sampling chamber such that the emitted light passes through the sample of the medicinal fluid within the fluid sampling chamber. The light emission may be directed through the imaging window of the fluid sampling chamber to interact with the medicinal fluid sample contained within the optical path. The method may include receiving, by a photodetector positioned on a second, opposite side of the fluid sampling chamber, light that has been exposed to one or more elements within the sample of the medicinal fluid within the fluid sampling chamber.

The DFC system may process data at a frequency between 0.1 Hz and 1 Hz to ensure accurate real-time monitoring of the medicinal fluid composition. The processing frequency range may provide sufficient temporal resolution for detecting concentration changes while maintaining adequate signal-to-noise ratios for accurate spectral measurements. The real-time processing capability may enable immediate feedback regarding the medicinal fluid composition during the analysis procedure.

The method may include generating, by at least one processing element, an optical absorption spectrum from the received light from the photodetector. The processing element may receive electrical signals from the photodetector and apply signal processing algorithms to extract spectral information from the received light. The processing element may correlate the received light with the optical wavelengths emitted by the DFC to reconstruct an absorption spectrum of the medicinal fluid sample.

The method may include identifying, by the at least one processing element, at least one of the one or more elements within the fluid sampling chamber exposed to the emitted light. The identification process may involve comparing the generated optical absorption spectrum against reference spectral databases to identify specific molecular components within the medicinal fluid. The system may include calibration and reference systems with standard samples and reference spectra for accurate identification and quantification of analytes in the flowing liquid.

The method may include drawing, via the syringe, the medicinal fluid from the fluid sampling chamber and pushing, via the syringe, the medicinal fluid back into the medicinal fluid delivery container after spectroscopic analysis is performed of the medicinal fluid. The fluid return process may ensure that the analyzed medicinal fluid sample is not wasted and remains available for therapeutic administration, thereby maximizing the utilization of expensive pharmaceutical formulations while providing quality control verification.

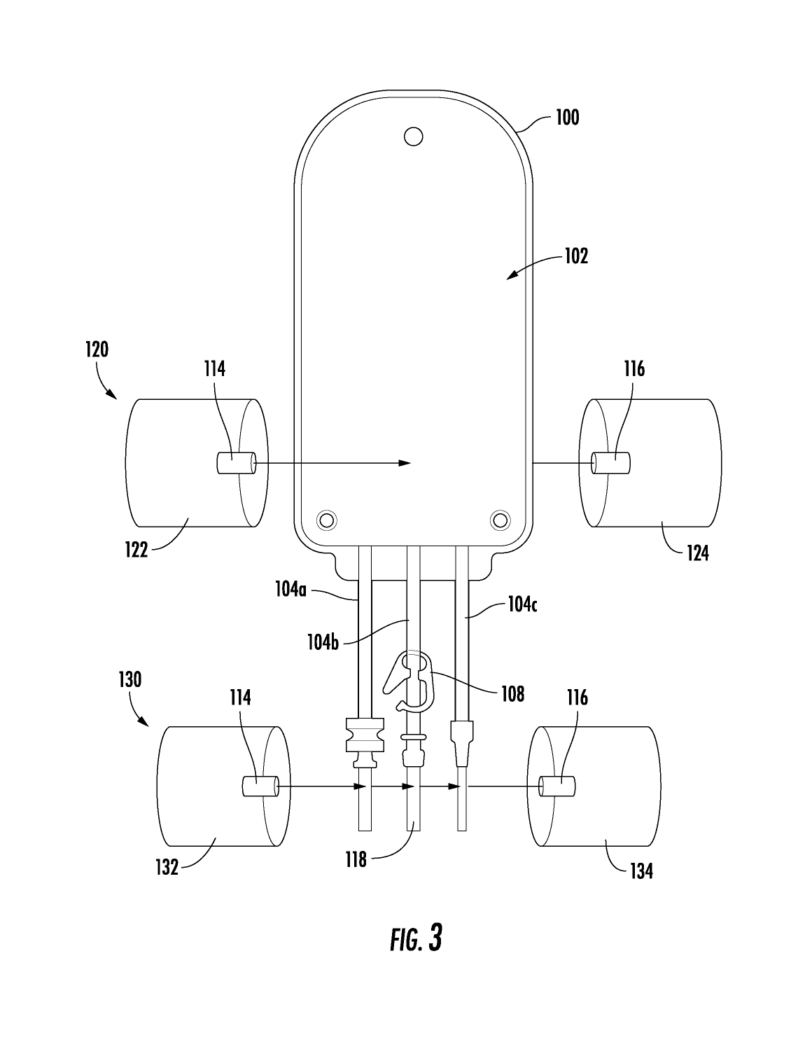

As illustrated in FIGS. 3-5, a system for in situ spectroscopic analysis of a medicinal fluid may employ a positioning structure configuration that enables direct analysis through medicinal fluid delivery containers without requiring separate sampling chambers or fluid transfer procedures. The device may comprise a positioning structure that provides mechanical support and spatial control for optical components during spectroscopic analysis operations. The positioning structure may be constructed from materials that provide stability and precision positioning capabilities while accommodating various sizes and configurations of medicinal fluid delivery containers.

A dual frequency comb (DFC) may be positioned on a first side of the positioning structure to provide optical radiation for spectroscopic analysis. The DFC may be mounted or integrated within the positioning structure to maintain precise spatial relationships with other optical components during analysis procedures. The positioning of the DFC on the first side of the positioning structure may enable controlled optical coupling into medicinal fluid delivery containers positioned within the device.

A photodetector may be positioned on a second, opposite side of the positioning structure and facing the DFC to detect optical signals transmitted through medicinal fluid samples. The photodetector may be aligned with the DFC to ensure proper optical coupling and signal detection during spectroscopic analysis procedures. The positioning of the photodetector on the second, opposite side of the positioning structure may create an optical path that passes through medicinal fluid delivery containers placed between the DFC and photodetector components.

The device may include at least one processing element that coordinates the operation of the optical components and performs spectral analysis calculations. The processing element may control the DFC operation, acquire signals from the photodetector, and execute algorithms for spectral data processing and compound identification.

The positioning structure may be adapted to position the DFC and the photodetector at a desired separation distance on opposite sides of a medicinal fluid delivery container containing a medicinal fluid. The positioning structure may include mechanical interfaces, clamps, or guides that accommodate medicinal fluid delivery containers of various sizes and configurations. The desired separation distance may be controlled through adjustable mechanisms within the positioning structure that enable precise positioning of the optical components relative to the medicinal fluid delivery container.

The desired separation distance may be one millimeter or less to ensure adequate optical interaction with the medicinal fluid while maintaining sufficient signal strength for accurate spectral measurements. The sub-millimeter separation distance may be achieved through precision positioning mechanisms that compress or constrain the medicinal fluid delivery container to establish controlled optical path lengths through the medicinal fluid.

The positioning structure may be adjustable to vary the separation distance between the DFC and the photodetector based on the specific requirements of different medicinal fluid delivery containers or analysis procedures. The adjustable positioning capability may enable optimization of the optical path length for different types of medicinal fluids or container materials, ensuring consistent analysis performance across various applications.

The DFC may be adapted to emit light that passes through the medicinal fluid delivery container and the medicinal fluid contained within the container. The DFC may generate coherent optical radiation across a broad spectral range that enables simultaneous analysis of multiple pharmaceutical compounds within the medicinal fluid. The light emission may be directed through the container material and medicinal fluid to interact with molecular components and generate characteristic absorption signatures. The photodetector may be adapted to receive the emitted light from the DFC that has been exposed to one or more elements within the medicinal fluid exposed to the emitted light.

The medicinal fluid delivery container may comprise an intravenous (IV) bag constructed from flexible materials that may be compressed or constrained by the positioning structure to establish controlled optical path lengths. In some cases, the medicinal fluid delivery container may comprise tubing in fluid connection with an IV bag, enabling analysis of medicinal fluids during administration or transfer procedures.

A wavelength range of the light emitted by the DFC may be selected based on a desired one or more medicines to be analyzed. The wavelength selection may optimize the spectroscopic sensitivity for specific pharmaceutical compounds while minimizing interference from container materials or excipients within the medicinal fluid formulation.

A method for in situ spectroscopic analysis of a medicinal fluid may utilize the positioning structure device to perform direct analysis through medicinal fluid delivery containers. The method may comprise positioning, using a positioning structure, a dual frequency comb (DFC) on a first side of a medicinal fluid delivery container and a photodetector on a second, opposite side of the medicinal fluid delivery container containing a medicinal fluid with a desired separation distance therebetween. The positioning process may involve placing the medicinal fluid delivery container within the positioning structure and adjusting the spatial relationships between the optical components to achieve the desired separation distance.

The method may include adjusting the positioning structure to vary the separation distance between the DFC and the photodetector based on the specific characteristics of the medicinal fluid delivery container or the analysis requirements. The adjustment capability may enable optimization of the optical path length for different container materials, fluid compositions, or measurement objectives.

The method may proceed with emitting light from the DFC such that the emitted light passes through the medicinal fluid delivery container and the medicinal fluid contained within the container. The light emission may be directed through the container material to interact with the medicinal fluid and generate characteristic optical absorption signatures corresponding to the molecular composition of the fluid. The method may include receiving, by the photodetector, light that has been exposed to one or more elements within the medicinal fluid exposed.

The method may include generating an optical absorption spectrum from the received light from the photodetector and identifying at least one of the one or more elements within the medicinal fluid exposed to the emitted light.

Referring to FIG. 3, a dual positioning mechanism configuration may enable simultaneous analysis of the fluid medicine 102 through multiple pathways, providing comprehensive monitoring capabilities for medicinal fluid delivery systems. The IV bag 100 may be configured with the first access port 104a, second access port 104b, and third access port 104c positioned at the bottom of the bag to enable fluid communication with downstream components. Each access port may connect to distal tubing 118 that extends downward from the IV bag 100 to facilitate fluid delivery and analysis operations.

A first positioning mechanism 120 may comprise a first clamping portion 122 and a second clamping portion 124 that are positioned to engage the IV bag 100 directly. The first clamping portion 122 and second clamping portion 124 may be configured to compress the IV bag 100 to establish a known distance between opposing surfaces of the bag material. The compression action may flatten the flexible container material to create a controlled optical path length through the fluid medicine 102 contained within the IV bag 100.

The first positioning mechanism 120 may enable spectroscopic analysis through the material of the IV bag 100 without requiring fluid sampling or transfer procedures. The DFC 114 and photodetector 116 may be integrated with the first positioning mechanism 120 to provide optical radiation and signal detection capabilities for analyzing the fluid medicine 102 directly through the bag material. The positioning mechanism may maintain precise spatial relationships between the optical components and the compressed IV bag 100 to ensure consistent measurement conditions.

A second positioning mechanism 130 may comprise a first clamping portion 132 and a second clamping portion 134 that are positioned to engage the distal tubing 118 extending from the IV bag 100. The first clamping portion 132 and second clamping portion 134 may be configured to secure the distal tubing 118 at a fixed distance while maintaining the structural integrity of the tubing during analysis procedures. The second positioning mechanism 130 may provide mechanical support and positioning control for analyzing the fluid medicine 102 as the fluid medicine 102 flows through the distal tubing 118.

The second positioning mechanism 130 may include channels within the first clamping portion 132 and second clamping portion 134 to hold the distal tubing 118 in position during spectroscopic analysis. The channels may be shaped to accommodate the cylindrical geometry of the distal tubing 118, providing secure positioning without deforming or occluding the fluid pathway. The channeled positioning mechanism may differ from flat clamping surfaces used for compressing flexible IV bag materials, as the channels may maintain the tubular structure of the distal tubing 118 while establishing controlled optical path lengths.

Additional DFC 114 and photodetector 116 components may be integrated with the second positioning mechanism 130 to enable spectroscopic analysis of the fluid medicine 102 as the fluid medicine 102 flows through the distal tubing 118. The optical components may be positioned on opposite sides of the distal tubing 118 to create a transmission-based measurement configuration that analyzes the fluid medicine 102 during active flow conditions.

The first positioning mechanism 120 and the second positioning mechanism 130 may be used in conjunction with each other or each may be used separately depending on the monitoring requirements.

The positioning structure may be adjustable to vary the separation distance between the DFC 114 and the photodetector 116 for both the IV bag analysis configuration and the tubing analysis configuration. The adjustable positioning capability may enable optimization of optical path lengths for different container materials, tubing dimensions, or fluid compositions, ensuring consistent analysis performance across various medicinal fluid delivery applications.

The medicinal fluid delivery container may comprise tubing in fluid connection with the IV bag 100, enabling analysis of the fluid medicine 102 during transfer or administration procedures. The tubing configuration may provide access to flowing medicinal fluids without interrupting the delivery process, enabling continuous monitoring of drug concentrations and quality parameters during patient treatment.

The device configuration may provide comprehensive analysis capabilities by combining direct analysis through the IV bag 100 material with flow-through analysis of the distal tubing 118. The dual analysis approach may enable verification of medicinal fluid composition both in storage and during active delivery, providing enhanced quality control and safety monitoring for pharmaceutical administration procedures.

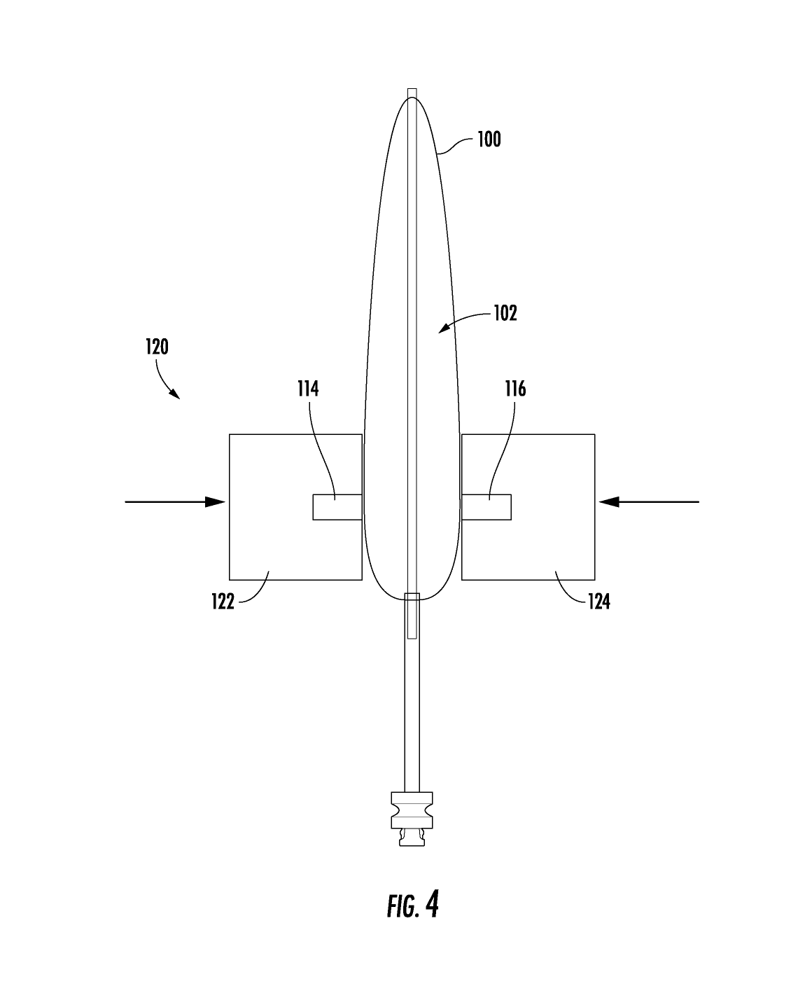

Referring to FIG. 4, a cross-sectional view illustrates the positioning mechanism 120 in position to compress the IV bag 100 and establish a consistent distance between the DFC 114 and photodetector 116 for spectroscopic analysis. The positioning mechanism 120 may include the first clamping portion 122 and second clamping portion 124 that apply compressive forces to opposing surfaces of the IV bag 100 containing the fluid medicine 102. The cross-sectional perspective demonstrates how the clamping portions may flatten the flexible container material to create a controlled optical path through the fluid medicine 102.

The first clamping portion 122 and second clamping portion 124 may apply pressure to compress the IV bag 100, reducing the optical path length through the fluid medicine 102 to establish the desired separation distance between the DFC 114 and photodetector 116. The compression action may deform the flexible bag material to create a flattened region where the fluid medicine 102 is constrained to a thin layer suitable for optical analysis. The positioning mechanism 120 may maintain a stationary and reproducible geometry during the compression process to ensure consistent measurement conditions across multiple analysis procedures.

The desired separation distance may be one millimeter or less to ensure adequate optical interaction between the emitted light and the fluid medicine 102 while maintaining sufficient signal strength for accurate spectral measurements. The sub-millimeter separation distance may be achieved through precise control of the compression force applied by the first clamping portion 122 and second clamping portion 124. The controlled compression may establish optical path lengths that enable detection of trace concentrations of pharmaceutical compounds within the fluid medicine 102 while minimizing signal attenuation through the sample.

The DFC 114 may comprise a PIC-scale DFC whose compact form factor facilitates integration with the first clamping portion 122 while maintaining precise optical alignment with the compressed IV bag 100 and the photodetector 116.

The positioning mechanism 120 may enable spectroscopic analysis of the fluid medicine 102 through the plastic membrane of the IV bag 100 without requiring direct contact with or sampling of the fluid medicine 102. The non-invasive analysis approach may preserve the sterility and integrity of the fluid medicine 102 while providing real-time verification of pharmaceutical compound concentrations and composition. The cross-sectional configuration demonstrates how the optical components may be integrated with the mechanical positioning elements to create a unified analysis system that combines precise distance control with advanced spectroscopic measurement capabilities.

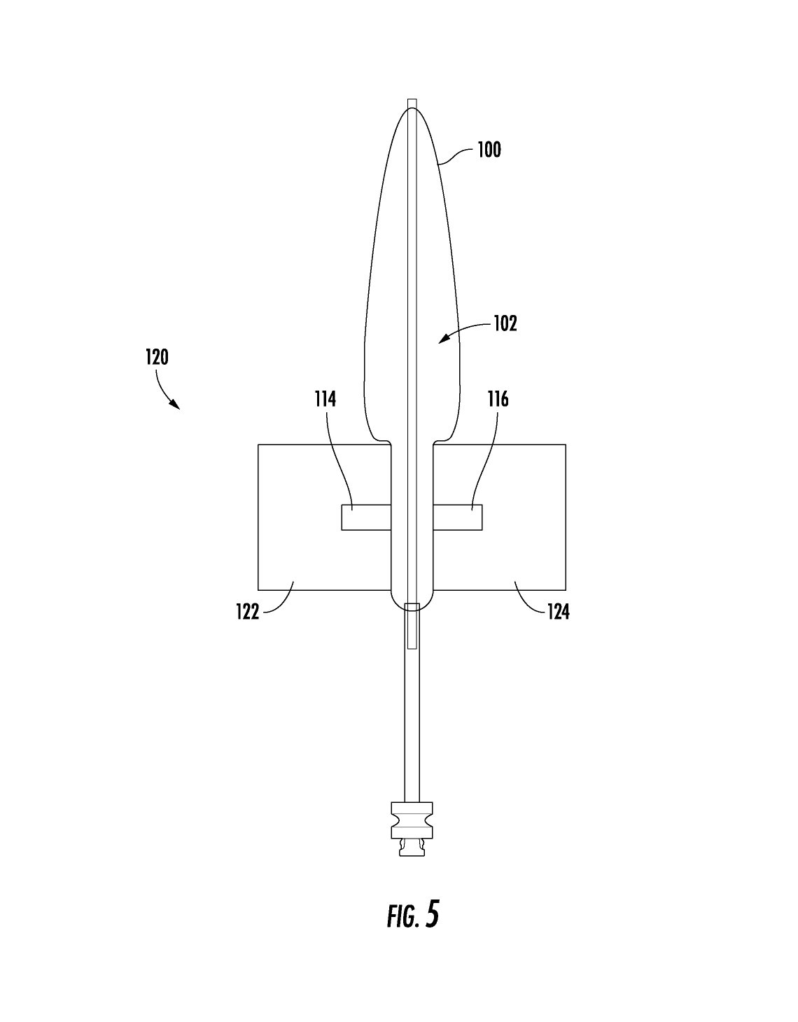

Referring to FIG. 5, a cross-sectional view illustrates the IV bag 100 positioned within the positioning mechanism 120 to demonstrate the spatial alignment and optical configuration during spectroscopic analysis of the fluid medicine 102.

A method for in situ spectroscopic analysis of a medicinal fluid may utilize a positioning structure configuration to perform direct analysis through medicinal fluid delivery containers without requiring fluid sampling or transfer procedures. The method may begin with positioning, using a positioning structure, a dual frequency comb (DFC) on a first side of a medicinal fluid delivery container and a photodetector on a second, opposite side of the medicinal fluid delivery container containing a medicinal fluid with a desired separation distance therebetween. The positioning process may involve placing the medicinal fluid delivery container within the positioning structure and engaging mechanical interfaces or clamps that secure the container in a fixed position relative to the optical components.

The method may proceed with emitting light from the DFC such that the emitted light passes through the medicinal fluid contained within the medicinal fluid delivery container. The method may include receiving, by the photodetector, light that has been exposed to one or more elements within the medicinal fluid. The method may include generating an optical absorption spectrum from the received light from the photodetector.

The processing element may receive electrical signals from the photodetector and apply signal processing algorithms to extract spectral information from the received light. The processing element may correlate the spectral information of the received light with the optical wavelengths emitted by the DFC to reconstruct an absorption spectrum of the medicinal fluid that reflects the molecular composition and concentration of pharmaceutical compounds. The spectrum generation process may involve Fourier transform analysis, digital signal processing techniques, or other mathematical algorithms that convert time-domain signals into frequency-domain spectral data. The processing element may apply calibration factors and reference corrections to account for variations in container materials, optical path lengths, or environmental conditions that may affect the measurement accuracy. The method may include identifying, by the at least one processing element, at least one of the one or more elements within the medicinal fluid exposed to the emitted light.

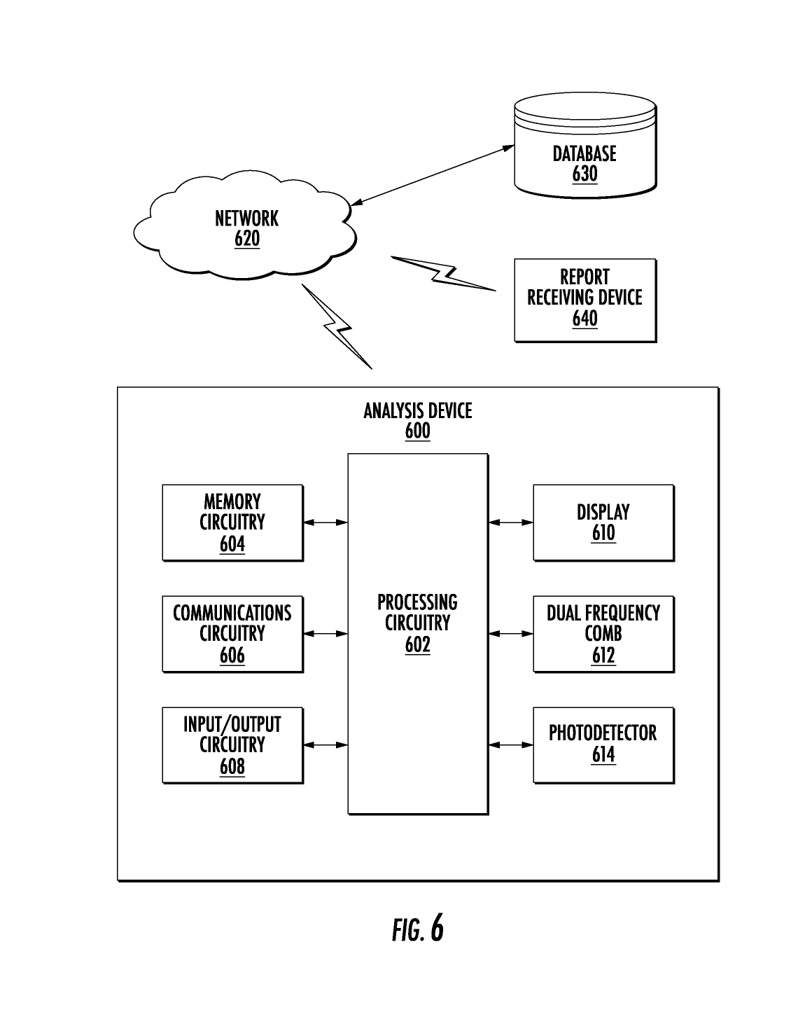

Referring to FIG. 6, an analysis device 600 may provide a comprehensive system architecture for coordinating spectroscopic analysis operations and managing data processing, communication, and user interface functions. The analysis device 600 may comprise several interconnected components that enable automated control of the spectroscopic measurement process while providing data management and reporting capabilities for healthcare applications.

The analysis device 600 may include processing circuitry 602 that serves as a central coordination component for managing the operation of the spectroscopic analysis system. The processing circuitry 602 may comprise microprocessors, digital signal processors, or specialized computational elements that execute control algorithms and data processing functions. The processing circuitry 602 may coordinate the timing and operation of optical components while simultaneously processing spectral data and executing identification algorithms for pharmaceutical compound analysis.

Memory circuitry 604 may be connected to the processing circuitry 602 to provide data storage capabilities for the analysis device 600. The memory circuitry 604 may store operational instructions, calibration parameters, reference spectral databases, and analysis results generated during spectroscopic measurement procedures. The memory circuitry 604 may comprise volatile memory elements such as random access memory for temporary data storage and non-volatile memory elements such as flash memory or solid-state drives for persistent data storage requirements.

Communications circuitry 606 may be connected to the processing circuitry 602 to enable data exchange between the analysis device 600 and external systems or devices. The communications circuitry 606 may support various communication protocols including wireless communication standards, ethernet connections, or specialized medical device communication interfaces. The communications circuitry 606 may enable remote monitoring capabilities and data sharing with healthcare information systems or pharmaceutical quality control databases.

Input/output circuitry 608 may be connected to the processing circuitry 602 to facilitate data transfer and user interactions with the analysis device 600. The input/output circuitry 608 may provide interfaces for connecting external devices, photodetectors, or control elements to the analysis device 600. The input/output circuitry 608 may enable healthcare providers to configure analysis parameters, initiate measurement procedures, or retrieve analysis results through various interface mechanisms.

A display 610 may be connected to the processing circuitry 602 to present information and analysis results to users of the analysis device 600. The display 610 may comprise liquid crystal displays, organic light-emitting diode displays, or other visual presentation technologies that enable real-time monitoring of analysis procedures and presentation of spectral data or identification results. The display 610 may provide graphical user interfaces that enable healthcare providers to monitor analysis progress, review spectral data, and access analysis reports during pharmaceutical quality control procedures.

A dual frequency comb (DFC) 612 may be connected to the processing circuitry 602 to provide optical frequency generation capabilities for spectroscopic analysis operations. The DFC 612 may generate coherent optical radiation across a broad spectral range with precisely controlled frequency spacing between adjacent spectral lines. The processing circuitry 602 may control the operation of the DFC 612, including timing, power levels, and spectral characteristics, to optimize the optical analysis conditions for specific pharmaceutical compounds or measurement requirements.

A photodetector 614 may be connected to the processing circuitry 602 to detect optical signals after the optical signals have passed through or interacted with medicinal fluid samples. The photodetector 614 may convert optical signals into electrical signals that may be processed by the processing circuitry 602 to extract spectral information. The photodetector 614 may record intensity measurements at each optical frequency generated by the DFC 612, enabling reconstruction of absorption spectra corresponding to the molecular composition of analyzed medicinal fluids.

The analysis device 600 may communicate with external components through a network 620 that facilitates data exchange and remote system integration. The network 620 may comprise local area networks, wide area networks, internet connections, or specialized healthcare communication networks that enable connectivity between the analysis device 600 and external data management systems. The communications circuitry 606 may interface with the network 620 to enable data transmission and reception capabilities for the analysis device 600.

A database 630 may be connected to the network 620 to provide centralized storage and management of reference spectral data, calibration parameters, and analysis results. The database 630 may store reference spectra corresponding to known pharmaceutical compounds, enabling the processing circuitry 602 to compare measured spectral data against established reference standards for compound identification. The database 630 may maintain calibration data that accounts for variations in optical components, environmental conditions, or measurement configurations that may affect analysis accuracy.

The database 630 may include calibration and reference systems with standard samples and reference spectra for accurate identification and quantification of analytes in medicinal fluids. The standard samples may comprise known concentrations of pharmaceutical compounds that serve as reference points for quantitative analysis procedures. The reference spectra may provide baseline spectral signatures for specific drugs, excipients, or contaminants that enable accurate identification of molecular components within complex medicinal fluid formulations.

A report receiving device 640 may be connected to the network 620 to receive analysis reports and data from the analysis device 600. The report receiving device 640 may comprise computer systems, mobile devices, or specialized healthcare information terminals that enable healthcare providers to access analysis results remotely. The report receiving device 640 may receive real-time analysis data, historical measurement records, or quality control reports generated by the analysis device 600 during pharmaceutical analysis procedures.

The network 620 may facilitate data exchange between the analysis device 600, database 630, and report receiving device 640, enabling distributed data management and remote monitoring capabilities for pharmaceutical quality control applications. The network connectivity may enable healthcare providers to access analysis results from multiple locations while maintaining centralized data storage and management through the database 630. The distributed system architecture may support quality control workflows that require coordination between multiple healthcare personnel or pharmaceutical preparation facilities.

The processing circuitry 602 may execute algorithms that compare measured spectral data against reference spectra stored in the database 630 to identify specific pharmaceutical compounds within analyzed medicinal fluids. The identification algorithms may utilize pattern matching techniques, spectral deconvolution methods, or machine learning approaches that recognize characteristic absorption features of pharmaceutical compounds. The processing circuitry 602 may determine concentration levels of identified compounds by comparing measured absorption intensities against calibrated reference standards stored in the database 630.

The analysis device 600 may generate comprehensive reports that include identification results, concentration measurements, quality control assessments, and recommendations for healthcare providers. The reports may be transmitted through the network 620 to the report receiving device 640, enabling real-time communication of analysis results to healthcare personnel responsible for pharmaceutical administration or quality control oversight.

The dual frequency comb spectroscopy systems for medicinal fluid analysis may provide numerous technical and clinical benefits that address challenges in pharmaceutical quality control and patient safety. The spectroscopic analysis approach may enable accurate dosaging and concentration verification of therapeutic compounds within medicinal fluids, reducing the risk of adverse effects and improving treatment efficacy for patient populations requiring precise pharmaceutical administration.

The systems may prevent infections by minimizing mistakes in the drug administration process for immunocompromised patients. The spectroscopic verification capabilities may reduce human error associated with manual monitoring and adjustment procedures that are prone to inaccuracies and time-consuming validation steps. The automated analysis approach may eliminate subjective assessments and provide objective measurements of pharmaceutical compound concentrations, thereby reducing the likelihood of dosing errors that may compromise patient safety.

The dual frequency comb spectroscopy technology may offer cost efficiency advantages by reducing dosing errors and improving treatment outcomes, which may lower healthcare costs associated with managing adverse effects and complications. The prevention of medication errors may reduce the need for corrective treatments, extended hospital stays, or additional medical interventions that result from improper pharmaceutical administration. The cost efficiency may be particularly beneficial for expensive therapeutic formulations such as chemotherapy agents where medication waste due to dosing errors represents significant financial losses.

The spectroscopic systems may provide enhanced performance compared to existing analytical methods by offering rapid analysis times, high spectral resolution, and the capability to simultaneously detect multiple compounds within a single measurement. The dual frequency comb approach may generate dramatic gains in data acquisition speed, spectral resolution, and sensitivity compared to conventional spectrometer systems that rely on mechanical scanning mechanisms. The enhanced performance characteristics may enable real-time analysis without requiring sample preparation or laboratory processing, thereby reducing analysis time and maintaining the chain of custody for medicinal fluids.

The point-of-care analysis capabilities may enable immediate verification of pharmaceutical formulations at the location where medicinal fluids are prepared or administered. The point-of-care approach may eliminate the need to transport samples to centralized laboratory facilities, reducing analysis turnaround times and enabling immediate decision-making regarding pharmaceutical administration. The real-time analysis capability may provide healthcare providers with immediate feedback regarding medicinal fluid composition, enabling prompt adjustments to treatment protocols when necessary.

The systems may minimize workflow impact on healthcare staff by providing integrated analysis capabilities that require minimal additional steps in existing pharmaceutical preparation and administration procedures. The automated analysis approach may reduce the time and effort required for manual quality control procedures while providing more comprehensive and accurate assessment of medicinal fluid composition. The minimized workflow impact may be particularly valuable in healthcare environments where staff time is limited and efficiency is paramount for patient care delivery.

The non-invasive analysis capabilities may preserve the sterility and integrity of medicinal fluids while providing comprehensive quality control verification. The non-invasive approach may eliminate the risk of contamination associated with sampling procedures and may enable analysis of the entire medicinal fluid volume without waste. The preservation of medicinal fluid integrity may be particularly important for expensive pharmaceutical formulations where sample loss represents significant cost implications.

The high sensitivity and precision of dual frequency comb spectroscopy may enable detection of trace concentrations of pharmaceutical compounds, making the technology suitable for applications requiring extremely accurate dosing such as neonatal drug administration. The precise concentration measurements may prevent overdose conditions in very low weight patients while ensuring therapeutic efficacy in vulnerable patient populations.

The systems may provide comprehensive quality control capabilities that verify the presence and concentration of active pharmaceutical ingredients, excipients, and potential contaminants within medicinal fluid formulations. The comprehensive analysis may detect degradation products, impurities, or formulation errors that may affect therapeutic efficacy or patient safety, enabling corrective actions before pharmaceutical administration.

While various embodiments in accordance with the principles disclosed herein have been shown and described above, modifications thereof may be made by one skilled in the art without departing from the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow, that scope including all equivalents of the subject matter of the claims. Each and every claim is incorporated as further disclosure into the specification and the claims are embodiment(s) of the present disclosure. Furthermore, any advantages and features described above may relate to specific embodiments but shall not limit the application of such issued claims to processes and structures accomplishing any or all of the above advantages or having any or all of the above features.

In addition, the section headings used herein are provided for consistency with the suggestions under 37 C.F.R. § 1.77 or to otherwise provide organizational cues. These headings shall not limit or characterize the disclosure set out in any claims that may issue from this disclosure. For instance, a description of a technology in the "Background" is not to be construed as an admission that certain technology is prior art to any disclosure in this disclosure. Neither is the "Summary" to be considered as a limiting characterization of the disclosure set forth in issued claims. Furthermore, any reference in this disclosure to "disclosure" or "embodiment" in the singular should not be used to argue that there is only a single point of novelty in this disclosure. Multiple embodiments of the present disclosure may be set forth according to the limitations of the multiple claims issuing from this disclosure, and such claims accordingly define the disclosure, and their equivalents, which are protected thereby. In all instances, the scope of the claims shall be considered on their own merits in light of this disclosure but should not be constrained by the headings set forth herein.

Also, systems, subsystems, apparatuses, techniques, and methods described and illustrated in the various embodiments as discrete or separate may be combined or integrated with other systems, modules, techniques, or methods without departing from the scope of the present disclosure. Other devices or components shown or discussed as coupled to, or in communication with, each other may be indirectly coupled through some intermediate device or component, whether electrically, mechanically, or otherwise. Other examples of changes, substitutions, and alterations are ascertainable by one skilled in the art and could be made without departing from the scope disclosed herein.

Many modifications and other embodiments of the disclosure set forth herein will come to mind to one skilled in the art to which these embodiments pertain having the benefit of teachings presented in the foregoing descriptions and the associated figures. Although the figures only show certain components of the apparatuses and systems described herein, various other components may be used in conjunction with the components and structures disclosed herein. Therefore, it is to be understood that the disclosure is not to be limited to the specific embodiments disclosed and that modifications and other embodiments are intended to be included within the scope of the appended claims. For example, the various elements or components may be combined, rearranged, or integrated in another system or certain features may be omitted or not implemented. Moreover, the steps in any method described above may not necessarily occur in the order depicted in the accompanying drawings, and in some cases one or more of the steps depicted may occur substantially simultaneously, or additional steps may be involved. Although specific terms are employed herein, they are used in a generic and descriptive sense only and not for purposes of limitation.

What is claimed is:

1. A device for in situ spectroscopic analysis of a medicinal fluid, the device comprising:
a positioning structure;
a dual frequency comb (DFC) positioned on a first side of the positioning structure;
a photodetector positioned on a second, opposite side of the positioning structure and facing the DFC;
at least one processing element;
wherein the positioning structure is adapted to position the DFC and the photodetector at a desired separation distance on opposite sides of a medicinal fluid delivery container containing a medicinal fluid;
wherein the DFC is adapted to emit light that passes through the medicinal fluid delivery container and the medicinal fluid;
wherein the photodetector is adapted to receive the emitted light from the DFC that has been exposed to one or more elements within the medicinal fluid exposed to the emitted light; and
wherein the at least one processing element as adapted to generate an optical absorption spectrum from the received light from the photodetector and identify at least one of the one or more elements within the medicinal fluid exposed to the emitted light.

2. The device of claim 1, wherein the desired separation distance is one millimeter or less.

3. The device of claim 1, wherein the DFC comprises a photonic integrated circuit (PIC)-scale DFC.

4. The device of claim 1, wherein the DFC comprises a fiber-based DFC.

5. The device of claim 1, wherein the medicinal fluid delivery container comprises an intravenous (IV) bag.

6. The device of claim 1, wherein the positioning structure is adjustable to vary the separation distance between the DFC and the photodetector.

7. The device of claim 1, wherein the medicinal fluid delivery container comprises tubing in fluid connection with an IV bag.

8. The device of claim 1, wherein the medicinal fluid comprises one or more cancer treatment medicine.

9. The device of claim 8, wherein the one or more cancer treatment medicines comprises one or more of a chemotherapy agent, an immunotherapy agent, a targeted therapy agent, and/or a hormonal therapy agent.

10. The device of claim 1, wherein a wavelength range of the light emitted by the DFC is selected based a desired medicinal fluid to be analyzed.

11. A method for in situ spectroscopic analysis of a medicinal fluid, the method comprising:
positioning, using a positioning structure, a dual frequency comb (DFC) on a first side of a medicinal fluid delivery container and a photodetector on a second, opposite side of the medicinal fluid delivery container containing a medicinal fluid with a desired separation distance therebetween;
emitting light from the DFC such that the emitted light passes through the medicinal fluid;
receiving, by the photodetector, the emitted light from the DFC that has been exposed to one or more elements within the medicinal fluid exposed to the emitted light;
generating, by at least one processing element, an optical absorption spectrum from the received light from the photodetector; and
identifying, by the at least one processing element, at least one of the one or more elements within the medicinal fluid exposed to the emitted light.

12. The method of claim 11, wherein the desired separation distance is one millimeter or less.

13. The method of claim 11, wherein the DFC comprises a photonic integrated circuit (PIC)-scale DFC.

14. The method of claim 11, wherein the DFC comprises a fiber-based DFC.

15. The method of claim 11, wherein the medicinal fluid delivery container comprises an intravenous (IV) bag.

16. The method of claim 11, further comprising adjusting the positioning structure to vary the separation distance between the DFC and the photodetector.

17. The method of claim 11, wherein the medicinal fluid delivery container comprises tubing in fluid connection with an IV bag.

18. The method of claim 11, wherein the medicinal fluid comprises one or more cancer treatment medicine.

19. The method of claim 18, wherein the one or more cancer treatment medicines comprises one or more of a chemotherapy agent, an immunotherapy agent, a targeted therapy agent, and/or a hormonal therapy agent.

20. The method of claim 11, wherein a wavelength range of the light emitted by the DFC is selected based a desired medicinal fluid to be analyzed.
